(19) United States
(12) Patent Application Publication
(10) Pub. No.: US 2006/0069349 A1
(43) Pub. Date: Mar. 30, 2006

(54) Method and device for delivering substance to tissue layers

(76) Inventors: Robert Ganz, Minnetonka, MN (US); Brian Zelickson, Minneapolis, MN (US); Mark Rydell, Golden Valley, MN (US)

Correspondence Address: Schwegman, Lundberg,;Woessner & Kluth, P.A., P.O. Box 2938, Minneapolis, MN, 55402, US

(21) Appl. No.: 11/230,304

(22) Filed: Sep. 19, 2005

Related U.S. Application Data
(63) Continuation of application No. PCT/US04/08077, filed on Mar. 17, 2004.
(60) Provisional application No. 60/455,452, filed on Mar. 18, 2003.
(60) Provisional application No. 60/502,914, filed on Sep. 15, 2003.

(51) Int. Cl. A61M 5/00

(52) U.S. Cl. 604/116

(57) ABSTRACT

A method and apparatus is described that includes a catheter and a penetrating member at a distal end of the catheter for penetrating a tissue. The penetrating member can have a lumen in communication with the catheter, the lumen having an opening at a surface of the penetrating member to deliver a substance through the lumen. The device includes means for determining a location of the opening relative to the tissue.

### CROSS-REFERENCE TO RELATED APPLICATIONS

This application is a Continuation Under 35 U.S.C. 1.111(a) of International Application No. PCT/US2004/008077 filed Mar. 17, 2004 and published in English as WO 2004/082491 A1 on Sep. 30, 2004, which claims the benefit under 35 U.S.C. 119 (e) of U.S. Provisional Application No. 60/455,452 filed on Mar. 18, 2003, and U.S. Provisional Application No. 60/502,914 filed on Sep. 15, 2003, which applications and publication are incorporated by reference in their entirety.

### FIELD

This application relates generally to a medical device and more specifically to a device and method to deliver a substance to tissue layers.

### BACKGROUND

In treating certain disease states, it is sometimes important to deliver medicines or materials into or through the wall of a viscus. For example, in patients with gastroesophageal reflux disease (GERD) it is sometimes necessary to place sutures or implants into or through the wall of the esophagus or stomach, in the area of the gastroesophageal junction, to prevent gastric acid from regurgitating into the esophagus.

Delivering medications or other materials into or through the appropriate part of the viscus wall can be difficult however, since the various layers of the wall can be quite thin and inadvertently traversed. The walls of most of the organs of the gastrointestinal tract, including the esophagus, are typically no more than 5-6 mm thick when non-distended, and can be as thin as 2-3 mm when distended. The walls of the gastrointestinal organs are composed of multiple layers including the mucosa, or innermost lining, the submucosa, or connective tissue layer, the muscle layer, and the adventitia. In the non-distended esophagus, the mucosa is approximately 1-2 mm, the submucosa is 1 mm, and the muscle layer is 1-2 mm.

Thus, in order to treat GERD, medication or material placed into the muscle or submucosal layer, for example, has to be positioned into a space no more than 1-2 mm to be of benefit to the patient. If the material is not placed in the correct position then it may not have the intended effect, or even worse may migrate out of the wall of the viscus and cause harm to the patient. The same is true for other conditions of the esophagus such as achalasia requiring botulinum toxin injection, or other conditions of the gastrointestinal tract.

One specific example of a material that has to be injected into the proper esophageal layer, at an exact depth, in order to be effective and safe, is a biopolymer made out of ethylene vinyl alcohol co-polymer sold under the trademark ENTERYX. ENTERYX is a substance that when injected into the esophageal muscle layer can prevent GERD. The product can work well when placed correctly i.e. into the muscle, but is ineffective when injected too superficially, and can be potentially hazardous if injected too deep i.e. through the wall of the esophagus. There is no available method or device that allows for the proper placement of ENTERYX into the appropriate tissue plane under direct vision. The current placement technique requires x-ray fluoroscopic guidance, which is expensive, inexact and exposes the operator to hazardous radiation.

Other treatments for GERD, such as collagen or hyaluronic acid with dextronomer, need to be placed into the submucosal space to be effective. If these substances are placed too deep, i.e. in the muscle layer, they will not work in the intended manner. Still other devices, such as endoscopic sutures and plicators need to be placed outside the esophageal or gastric wall to be effective.

Thus a need exists for a method and device that would allow for the exact positioning of material or medication, specifically ENTERYX but also including other substances, into the appropriate space in the wall of a viscus, or outside the wall of a viscus.

### SUMMARY

In one aspect, an apparatus includes a catheter, a penetrating member at a distal end of the catheter for penetrating a tissue, an opening at a surface of the penetrating member to deliver a substance, and means for determining a location of the opening relative to the tissue.

In one aspect, a method includes inserting a catheter into a body, the catheter having a penetrating member on a distal end of the catheter, delivering a fluid through the catheter and the penetrating member and monitoring a flow rate of the fluid or a pressure of the fluid, inserting the penetrating member into a tissue, and injecting a therapeutic material into the tissue through the penetrating member when it is determined, using the flow rate or the pressure, that a distal opening of the penetrating member is located within the tissue.

### BRIEF DESCRIPTION OF THE DRAWINGS

FIG. 1A shows a schematic view of a device according to one embodiment.

FIGS. 1B-1D show details of the device of FIG. 1A.

FIGS. 1E-1K shows various catheters and penetrating members according to various embodiments.

FIGS. 2A-2C show a device according to one embodiment.

FIG. 3 shows a device according to one embodiment.

FIG. 4 shows a device and a pressure measurement device according to one embodiment, in an esophagus.

FIG. 5 shows a device according to one embodiment.

FIG. 6 shows a device and an injection mechanism according to one embodiment.

FIG. 7 shows a device according to one embodiment.

FIG. 8 shows a device according to one embodiment.

FIG. 9 shows a device according to one embodiment.

FIG. 10 shows a device in an endoscope, according to one embodiment.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

In one embodiment, the current device is designed to locate and identify individual layers of the wall of a viscus, and to facilitate placement of a substance, material, or medication into a specific layer, or through the wall, for treatment of a specific disease or condition. Although the device can be used for any number of substances, materials, or medications for treatment of various disease states or conditions, one application of the device is to facilitate placement of ENTERYX into the muscular layer of the esophageal wall in order to treat GERD.

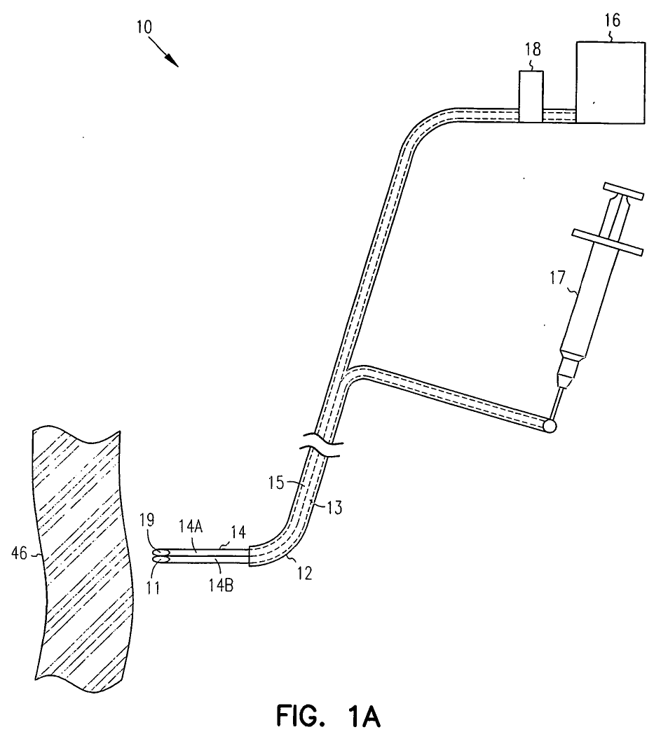

FIG. 1A shows a system 10 to deliver a material or substance to a specific layer of a viscus, in accordance with one embodiment. System 10 includes a catheter 12 and a penetrating member 14, such as a needle, coupled to the distal end of catheter 12. In this embodiment, catheter 12 is a flexible catheter and includes a first lumen 13 and a second lumen 15, and the penetrating member 14 includes two separate penetrating members, such as needles 14A and 14B, with a lumen of each needle 14A and 14B, respectively, in communication with one of the lumens of the catheter 12. Each lumen of the catheter extends to an opening 11, 19, on a surface of the needles 14A and 14B. In some embodiments, penetrating member 14 can be an integral portion of catheter 12, formed by molding, for example. Other embodiments use metal or plastic needles that can be bonded to the catheter by glue or adhesive, for example. In one example, penetrating members 14A and 14B are 23 or 25 gauge needles and catheter 12 has an outside diameter of about 2.5 mm.

In this example, the second lumen 15 is in communication with a fluid delivery device 16, such as a pump. Fluid delivery device 16 delivers a constant flow of a fluid, which can be a liquid or a gas, such as air, CO₂, or helium, for example, through second lumen 15. In one embodiment, the fluid-delivery lumen 15 incorporates a member 18 to determine a rate of flow of fluid through the second lumen 15. For example, member 18 can be a flow-meter to indicate the fluid-flow through the lumen. In other examples, to be discussed below, member 18 can be a pressure measurement device to measure the pressure of the fluid within the lumen.

Delivery device 16 can be set to deliver a constant flow of CO₂ (or other fluid) through the lumen 15, and by monitoring the flow-meter, a user is able to determine if opening 19 of penetrating member 14A is located within tissue 46 or not since when the opening 19 is within the tissue, the flow is reduced to a level so as to be virtually stopped (although a small amount may still flow into the tissue), and when the opening 19 is through the tissue, the flow restarts. In one embodiment, member 18 can be a KFR flow-meter by Kobold Instruments. In other embodiments, fluid flow can be determined using a bubble tube, a penetrating member gauge, a solid-state device having a digital or analog output, or a sound or light output to alert the user of fluid flow, for example.

In one example, a substance such as a therapeutic material, for example, the ethylene vinyl alcohol co-polymer sold under the trademark ENTERYX, can be delivered through lumen 13 of catheter 12. The position of the opening 19 on penetrating member 14A can be determined using the fluid-delivery lumen 15 by determining if the flow rate is reduced or stopped. When the penetrating member is properly positioned, the ENTERYX or another substance can be injected through the first lumen 13 and opening 11. An injection device 17, such as a syringe, can be at a proximal section of the catheter and used to deliver the therapeutic material to tissue 46.

In this embodiment, openings 11 and 19 are at approximately the same location on penetrating members 14A and 14B (i.e., the openings co-terminate at the tips of their respective penetrating members). Accordingly, knowing the depth of opening 19 gives the user knowledge of the location of opening 11. As will be discussed below, in some embodiments the openings can be staggered relative to each other. For example, opening 19 can be on the tip and opening 11 can be 1 or 2 mm back, for example, or vice versa. In one example use of system 10 the injection can repeated about 4 times or more.

In use, a fluid flow through the fluid-delivery lumen 15 is begun using device 16 or other flow means. When the fluid is flowing through the lumen the flowmeter 18 indicates fluid-flow. The catheter is placed in the lumen of the esophagus and fluid flow continues through the lumen 15. Referring to FIGS. 1B-1D, the catheter is advanced and penetrating member 14 pricks the tissue 46 (FIG. 1B), stopping the fluid-flow. It is then known that the penetrating member is in the tissue. In one example, a substance, such as a therapeutic material is then delivered through lumen 13 and penetrating member 14B into tissue 46 via opening 11.

An endoscope (not shown) can then be used to detect if a bleb and darkening forms during injection of the therapeutic material. If such a bleb and darkening does form, the user knows the substance is being injected too superficially relative to the tissue. The injection is stopped and the penetrating member is advanced further into the tissue while the air-flow is monitored. If the air flow remains stopped, but no bleb or darkening is seen, the user knows that the ENTERYX injection is correctly positioned (in the muscle layer) and can complete the injection. If the penetrating member is advanced too far (FIG. 1C), then the user knows the penetrating member is through the tissue because the flow will start again through penetrating member 14A. Then the penetrating member 14 is pulled back until the fluid-flow stops again. (FIG. 1D). At that point, it is known that the tip of the penetrating member is located in the tissue. The ENTERYX or other substance is then injected through the lumen 13 and penetrating member 14B into the tissue.

Again, if no bleb or darkening forms, the user knows the injection is in the correct location for the ENTERYX. After the injection, the penetrating member is pulled out and the process is repeated for other points in the tissue. Table 1, below shows the basic process that can be used to deliver a substance, such as ENTERYX, to a desired non-superficial location.

TABLE 1

| | No Fluid-flow |
|---|---|
| Bleb formation | Stop injecting and |
| | insert deeper |
| No bleb formation | Correctly positioned- |
| | finish treatment. |

Thus, a user positions the device until there is virtually no fluid flow as explained above. An injection is begun and if there is bleb formation, the user stops injecting and repositions the device. When there is both no fluid flow and no bleb formation, the device is properly positioned for an injection of the ENTERYX.

By using separate needles 14A and 14B, a constant flow of fluid can be delivered through one of the needles while the other is used to deliver the therapeutic material. Since the fluid is always flowing out, it is difficult for the therapeutic material, such as ENTERYX, to get into the other lumen and clog it. The constant flow of fluid flushes the lumen and keeps the penetrating member clear.

In further options, the device of system 10 can include a single lumen catheter 12 and/or single lumen penetrating member 14. For example, a single lumen can first be used to determine fluid-flow and then can be used to deliver the substance to the tissue. Some embodiments include multi-lumen catheters. In some embodiments, a double lumen catheter can be used with a single or double lumen penetrating member 14.

FIGS. 1E-1K show various embodiments of penetrating member/catheter combinations. FIG. 1E shows a device having a double lumen catheter 12 and a pair of penetrating members 14A and 14B which are in a staggered configuration such that opening 19 is distal from opening 11 by 1, 2, 3, mm or more. This device can be used by putting flow through penetrating member 14B and a substance through penetrating member 14A, or vice-versa. As one example, by inserting the penetrating member until flow through penetrating member 14B stops, the user knows that opening 19 is about 1-3 mm farther forward within the tissue and therapeutic material can be delivered through opening 19. Alternatively, flow can be delivered through penetrating member 14A and opening 19. When the penetrating member is advanced all the way through the tissue (See FIG. 1C), then flow will start again through opening 19, but opening 11 will still be within the tissue and a substance, such as a therapeutic material, can be delivered through opening 11 into the appropriate space.

FIG. 1F shows an example with a double lumen catheter 12 and a double lumen penetrating member 14C. FIG. 1G shows a double lumen catheter and a double lumen penetrating member 14D having staggered opening 19, 11. The embodiment of FIG. 1G can be used similarly as the embodiment of FIG. 1E.

FIG. 1H shows an example of a single lumen catheter 12B and a single lumen penetrating member 14E. FIG. 1J shows an example of a double lumen catheter 12 and a single lumen penetrating member 14E. FIG. 1K shows a coaxial double lumen catheter 12C and a coaxial double lumen penetrating member 14F. There could also be a single lumen tube for flow inside another tube used for therapeutic injection or vice versa, using a single or double lumen penetrating member.

In other embodiments, these various designs can be used in different configurations. Also, the catheter and penetrating member lumens can be symmetrical or asymmetrical. In some embodiments, the penetrating member and the catheter can be hollow or solid, and the medium or fluid inside the catheter can be a gas or liquid including air, CO₂, helium, DMSO, water, saline, ENTERYX, etc., for example.

In other embodiments, the present device or the embodiments discussed herein and below can be used for delivery of other substances into or through the wall of a viscus. For example, the device can deliver a substance to the submucosa. In these embodiments, the user requires bleb formation to know the substance is correctly located. For example, collagen and hyaluronic acid with dextronomer need to be positioned in the submucosa to prevent reflux. To deliver such substances with the present system, a user advances the penetrating member until the opening is within the tissue, as discussed above. The substance is delivered and if a bleb forms, the user know the substance is being delivered correctly. If no bleb forms, then the substance is being delivered too deep. In that situation, the user pulls the penetrating member more superficially and (if there is still no flow) delivers the substance again until a bleb is seen. Thus, an endoscope can be used to determine if the device is correctly positioned to deliver a substance to an appropriate layer, whether that layer is deep (e.g. ENTERYX) or superficial (e.g. collagen).

In some embodiments, the device can deliver a substance through the wall of a viscus. For example, the penetrating member can be advanced into the tissue (where the user sees virtually no flow) and then through the tissue (flow). When the user sees the flow restart, then he or she knows that the opening of the device is through the tissue. Then substances such as clips, sutures, anchoring bars, plugs, plicators, etc. can be delivered through a lumen of the catheter and can be placed through the wall of the viscus or on the outside of the wall, for example.

In one embodiment, a device according to the present system can include a penetrating member, such as a needle, attached to a catheter that can measure pressure in the wall or through the wall of a viscus, such as the esophagus. The penetrating member and catheter can measure either positive or negative pressure depending on its location in or through the wall. For example, the esophagus and esophageal wall is located in the chest, or more specifically, the mediastinum, of humans and animals. Pressures in the mediastinum and chest cavity tend to be negative due to the expansion of the chest cavity and lungs, and descent of the diaphragm during the act of breathing. With each breath the space surrounding the esophagus expands, creating net negative pressure in the chest and area outside the esophagus and net positive pressure in the abdomen.

Thus if the penetrating member is through the esophageal wall it will register as negative pressure. If the penetrating member is in the esophageal wall it will register as zero pressure depending on its location, and if the penetrating member is in the lumen of the esophagus the pressure recording will again be negative. Thus by a combination of direct vision (e.g. bleb formation and darkening for ENTERYX) and pressure recording one can reliably determine the location of the penetrating member, and can then reliably administer the appropriate substance, material, or medication into the proper space either through the penetrating member or by a different manner. Again, the device can also be used to deliver substances into a superficial location of the viscus, a deep location of the viscus, through the wall of the viscus, or on the outside of the viscus. If, the substance is being delivered to a superficial location, then the user requires bleb formation to know the substance is correctly located.

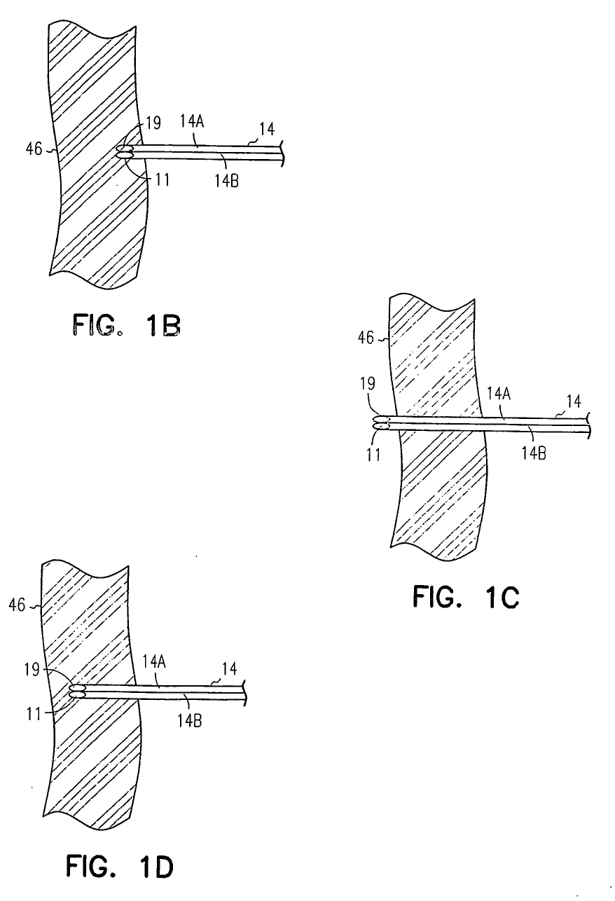

FIGS. 2A-2C show a system 30 according to one embodiment. System 30 includes a double lumen catheter 32 and a penetrating member 34 coupled to the distal end of the catheter and in communication with both lumens 31, 33 of catheter 32. In this embodiment, the lumens are side by side. In other examples, a catheter having coaxial lumens can be used. In this example, a therapeutic material, such as ENTERYX, is located in lumen 31 of catheter 32 and a second material, such as dimethyl sulfoxide (DMSO), is located in the other lumen 33. In one embodiment, the DMSO-filled lumen is coupled to a delivery mechanism, such as a syringe 36, and a container 38 with a 3-way stopcock 39 therebetween. Container 38 can be pressurized by a syringe 40 or other means. A pressure sensor or pressure gauge 42 is coupled to container 38 to measure the pressure in the container, and thus in lumen 33 of the catheter when the stopcock 39 is open.

In this example, DMSO is used to identify the pressure at an opening 35 near the tip of penetrating member 34. DMSO works well because its specific gravity is close to water. A substance such as ENTERYX is delivered through the other lumen 31. This provides a double lumen catheter, with different materials in each lumen, one for pressure sensing and clearing, the other for therapeutic injection. Some examples can use a different fluid, such as water or saline instead of DMSO. However, DMSO does not precipitate the ENTERYX such as water can. Instead, DMSO keeps the ENTERYX in solution. Catheter 32 can be formed of a DMSO-compatible material such as polypropylene, polyethylene, nylon, acetal, and polyvinyl chroride. Some embodiments use a fluoroplastic.

The penetrating member position can be determined using the DMSO lumen as a pressure identifier. When the penetrating member is properly positioned, ENTERYX is injected through the other lumen. The DMSO lumen can then be used to clear the penetrating member when necessary. In one example use of system 30 the injection can repeated about 4 times or more.

Thus, in use the catheter is advanced and pricks the tissue 46 (FIG. 2B). The container 38 is then pressurized using syringe 40. The stopcock 39 is then opened so that pressure gauge 42 sees the pressure at the opening 35 of penetrating member 34. If the penetrating member 34 is then pushed further into and through tissue 46 the pressure drop will be noted on gauge 42 (FIG. 2C). Then the penetrating member 34 is pulled back until the pressure stops dropping. At that point, it is known that the opening 35 near or at the tip of penetrating member 34 is properly located in the tissue for ENTERYX delivery. The ENTERYX is then injected through the second lumen into the tissue 46. After the injection, the penetrating member can be flushed by DMSO if necessary using syringe 36 and the process is repeated for other points in the tissue.

Again, some embodiments can deliver a substance to a superficial location, a deep location, a location through the wall of the viscus, or on the outside of the viscus. As discussed above, an endoscope (not shown) can be used to detect if the material is in the appropriate tissue layer. For example, if a bleb forms during injection of ENTERYX, the user know the substance is being delivered too superficially. Alternatively, for a substance such as collagen, the user needs to see a bleb to know that the substance is being delivered appropriately.

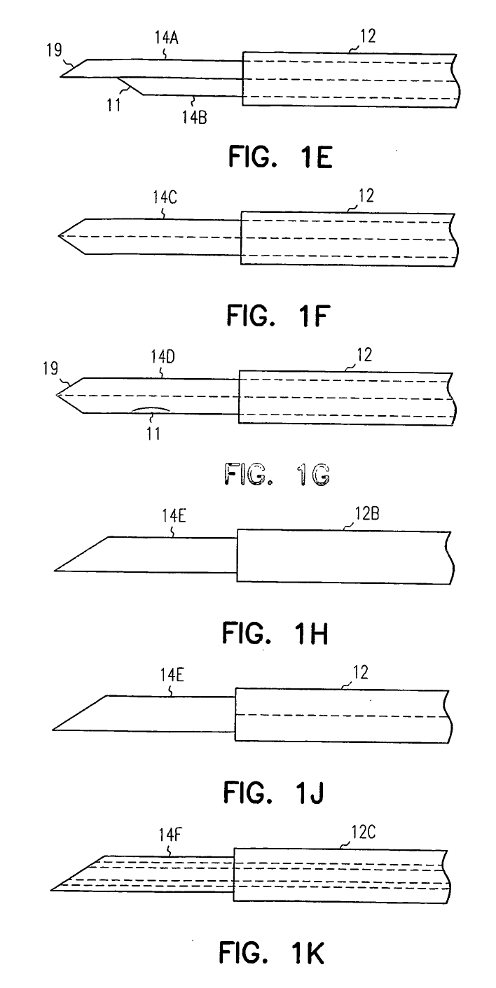

FIG. 3 shows a catheter 52 according to one embodiment. In this example, catheter 52 includes a double lumen configuration having lumens 54 and 56. A penetrating member 58 coupled to the distal end of the catheter also includes a dual lumen design with each penetrating member passage only in communication with one of the catheter lumens. This prevents the mixing of the material in the catheter lumens or needle lumens. The dual lumen penetrating member 58 can be used for any embodiments discussed herein.

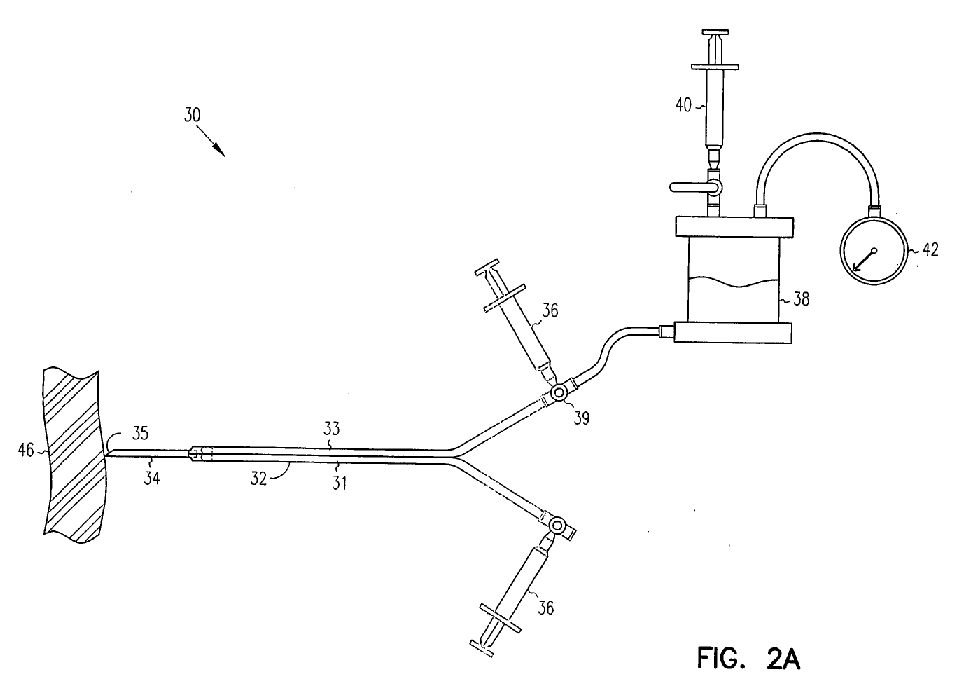

FIG. 4 shows a device 64 according to one embodiment. Device 64 includes a catheter 67 having a penetrating member 66 on a distal end of the catheter. Penetrating member 66 is penetrating through the wall 61 of the esophagus 72 near the esophago-gastric junction 65, into the chest cavity.

In one embodiment, device 64 includes a hollow penetrating member 66 at the distal position, attached to a hollow catheter 67, which is in turn connected to a pressure sensor or pressure gauge, such as a pressure recording mechanism 68 at the proximal position. The penetrating member 66 and catheter 67 conduct the pressure back to the recording mechanism 68 wherein the pressure can be measured and read in either analog or digital fashion. In this embodiment both the penetrating member and catheter are hollow, however, they can both be solid, or one can be hollow and the other solid. In this embodiment the hollow space in the penetrating member and catheter is filled with air, however, the space can also be filled with liquid (e.g. water), or ENTERYX. Thus the pressure can be conducted by a column of air or a column of fluid. In the case where the penetrating member and/or catheter are solid the pressure can be conducted by a solid state recording mechanism.

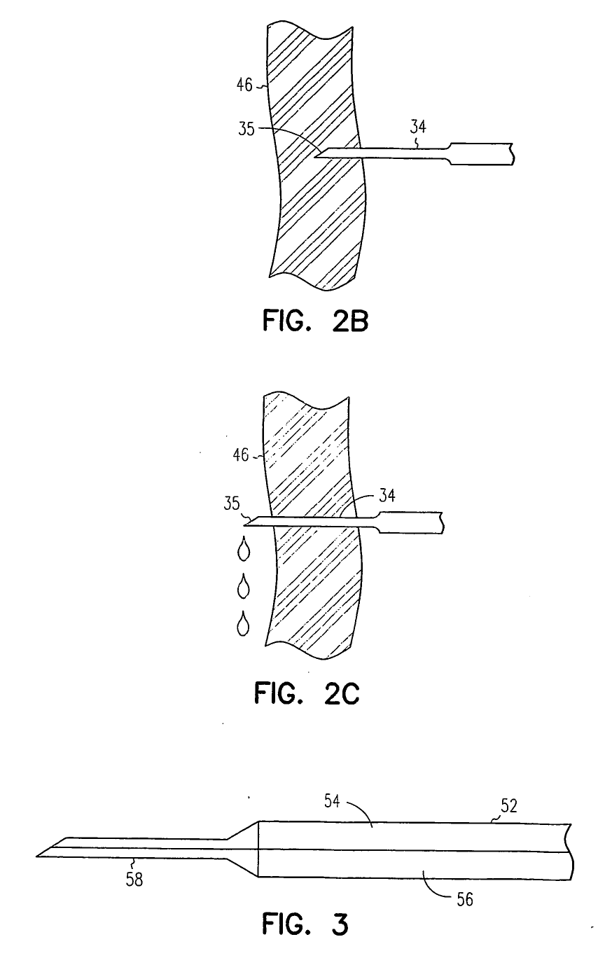

As shown in FIG. 5, which is not to scale, but is enlarged for clarity, the wall of the esophagus 72 is composed of the muscle layer 69, the submucosa 70, and the mucosa 71. Each specific layer in the wall, or the lumen, or the space outside the wall can be identified by a combination of vision and pressure recording, or by effect on the space by injecting fluid i.e. bleb formation. Thus the exact location of the penetrating member in each space can be readily determined.

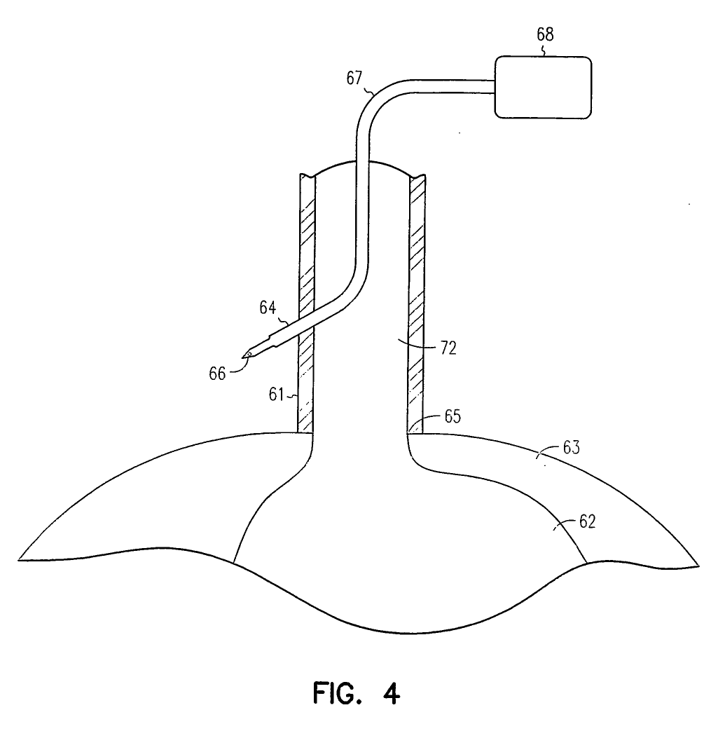

As shown in FIG. 6, in one embodiment the pressure can be measured alone, or a second attachment 73 can be made to the catheter to allow injection of material or ENTERYX 74 into the appropriate space, for example the muscle layer. The injecting mechanism 75 can be a syringe or any other type of mechanism that would allow for injection. It is also possible to have a single attachment to the catheter and first measure pressure and then switch the recording mechanism to the injecting mechanism through the same attachment. Thus pressure recording and injecting can either be concurrent or sequential.

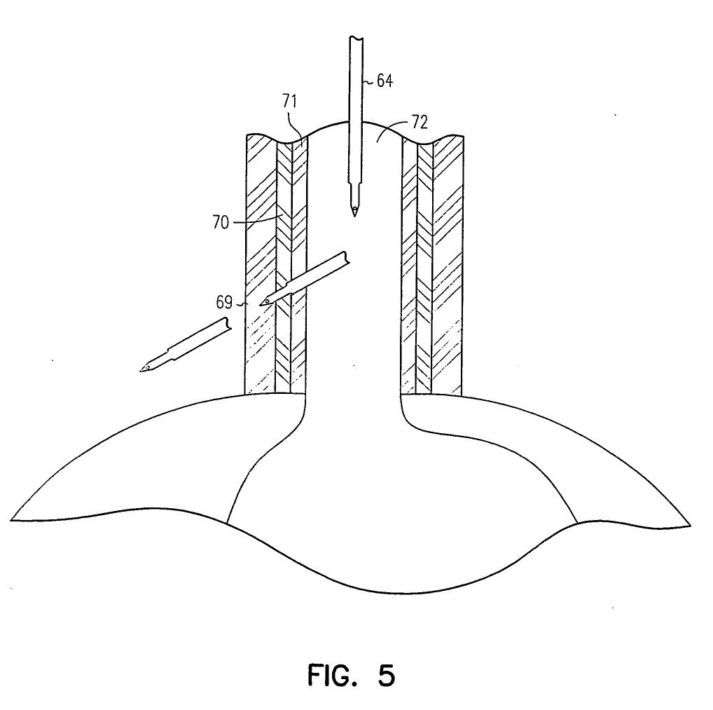

In another embodiment as shown in FIG. 7, the penetrating member 66 and catheter 67 can each have a double lumen to facilitate simultaneous pressure monitoring and injection. In this embodiment the pressure is monitored and recorded through one lumen via lumen opening 77, and the substance, such as ENTERYX 74 can be injected concurrently through the other lumen via lumen opening 76. In this arrangement at the proximal end, one lumen is attached to the pressure recording mechanism 68, and the other lumen is attached to the injecting mechanism 75 (FIG. 6).

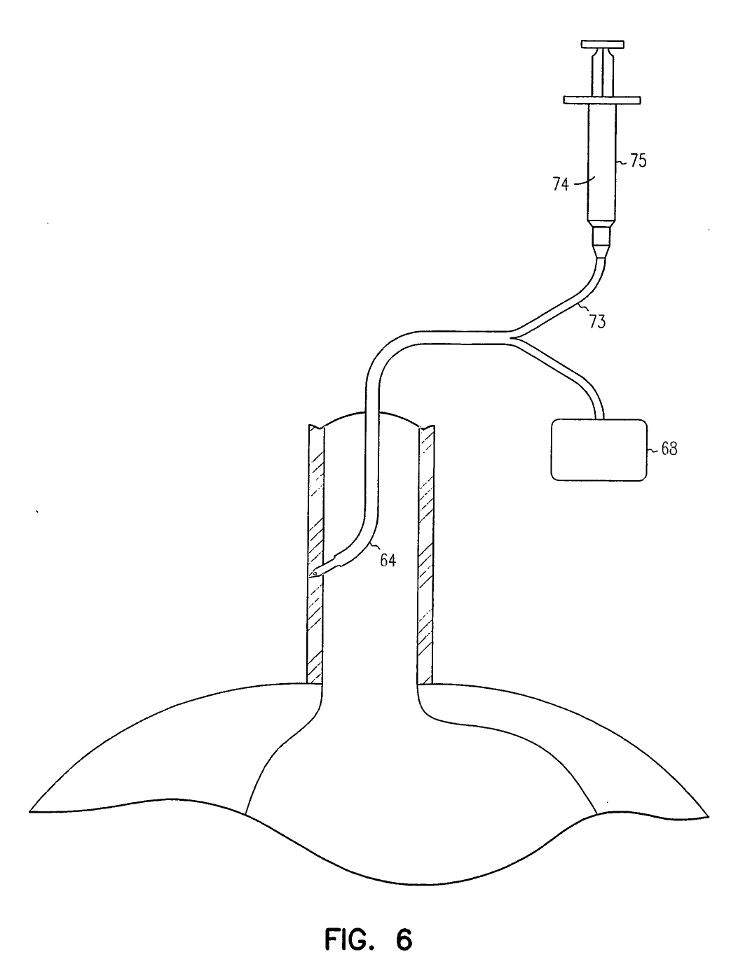

In one embodiment, as shown in FIG. 8, a pressure sensor 87 can be attached to the penetrating member tip to facilitate recording of the pressure. This sensor can be attached to either a single or double lumen hollow penetrating member and catheter to facilitate concurrent injection, or a solid penetrating member and catheter. The pressure from the sensor can be transmitted to the pressure recording mechanism by any manner of ways i.e. radio waves etc. in a fashion known in the art. In this embodiment, the sensor 87 can also be built into, or otherwise incorporated into, the tip or body of the penetrating member 66, or catheter 64; this can also include a solid state mechanism.

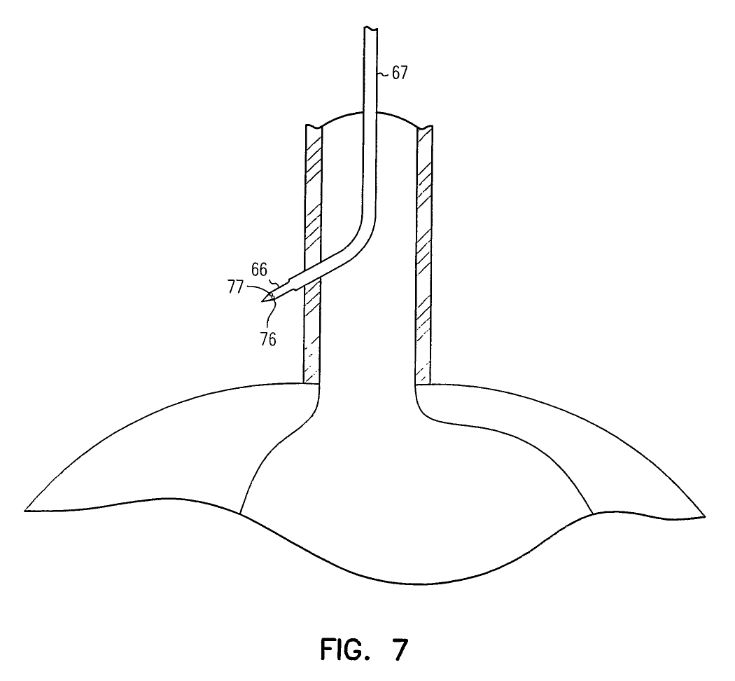

FIG. 9 shows one embodiment of a device. In this embodiment a pressure sensing solid catheter or probe 98 can be extended through one lumen 88 of a hollow penetrating member for measurement of pressure, concurrently with injection through a second lumen via an opening 89.

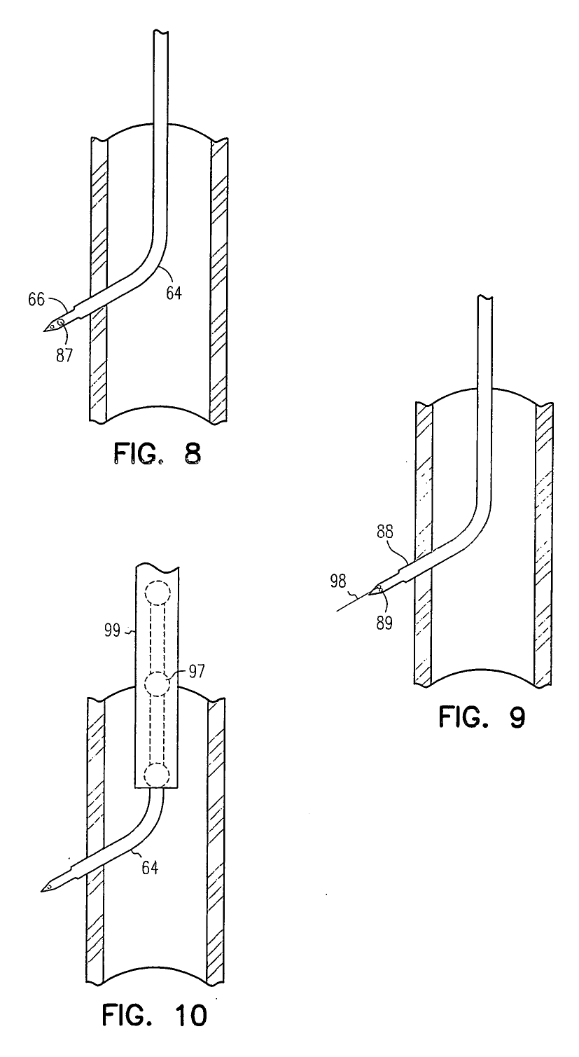

In any of the embodiments discussed herein, the penetrating member and catheter can be inserted under direct vision through an endoscope 99 and endoscope channel 97 during endoscopy as shown in FIG. 10, or without endoscopic guidance.

In some embodiments, the catheter can be passed over a guidewire, either in conjunction with an endoscope or without. Some embodiments can also be used with or without fluoroscopy, also with or without the concurrent use of an endoscope. Moreover, in some embodiments the penetrating member and catheter can also be attached to the outside of the endoscope for passage into the esophagus, by any manner of adhesive, strap, clip, etc. Accordingly, an endoscope can be used to determine if the device is correctly positioned to deliver a substance to an appropriate layer, whether that layer is deep (e.g. ENTERYX) or superficial (e.g. collagen).

Thus, in one or more embodiments, the present system provides a method and device for locating and identifying the individual layers of the walls of a viscus in order to facilitate placement of medication and material into the appropriate layer, through the wall, or outside the wall. In one example, the device can include a penetrating member attached to a catheter which is connected to a pressure recording mechanism, capable of determining either positive or negative pressure, or a flow monitoring device to determine flow through the catheter. In some embodiments, the material to be injected is ENTERYX and the target layer is the muscle layer of the esophageal wall. In some embodiments, the penetrating member and catheter can be extruded through the wall of the viscus to facilitate localization. In some embodiments, the device can deliver a substance to the submucosa. For example, collagen and hyaluronic acid with dextronomer need to be positioned in the submucosa to prevent reflux. To deliver such substances with the present system, a user advances the penetrating member until the opening is within the tissue, as discussed above. The substance is delivered and if a bleb forms, the user know the substance is being delivered correctly. If no bleb forms, then the substance is being delivered too deep. In that situation, the user pulls the penetrating member outward and (if there is still no flow) delivers the substance again.

In some embodiments, the device can be used to deliver a substance through the wall of a viscus and deliver substances such as clips, sutures, anchoring bars, plugs, plicators, etc. which can be delivered through a lumen of the catheter and be placed through the wall or on the outside of the wall, for example.

In some embodiments, the penetrating member and the catheter can be hollow or solid, and the medium inside the catheter can be gas or liquid including air, CO₂, helium, DMSO, water, saline, and ENTERYX, for example. In some embodiments, the penetrating member and catheter can include a solid state pressure sensing mechanism. In some embodiments, the pressure sensing device can be attached to or incorporated into the tip or body of the penetrating member.

In some embodiments, the device can be utilized in almost any organ of a body, hollow or solid, including but not limited to the gastrointestinal tract, genitourinary tract, the cardiac system, the vasculature, the respiratory system, the skin, and the skeletal muscle.

### EXAMPLE

A trial using the embodiment of FIG. 1A was done. In the trial, the ENTERYX polymer was localized into the deep esophageal layers (i.e. the muscle or deep submucosa adjacent to the muscle). Endoscopy was performed on 5 consecutive pigs. At 5 cm, 10 cm, and 15 cm above the GEJ, 4 circumferential 1 cc injections of ENTERYX were made (60 total) using a device such as described above in FIG. 1A, without fluoroscopy. Injections were assessed as superficial, deep into the esophageal wall, or transmural. Injections deemed transmural or superficial were repeated until the endoscopist felt that a deep injection had been made. The procedure was timed.

A blinded pathologist sacrificed each animal and harvested the esophagus. The mediastinal cavity and contiguous organs were searched for any extravasated ENTERYX material. The esophagus was examined grossly for ENTERYX adherent to the outside wall. The esophagus was then fixed in formalin and serially sectioned to identify the location of the ENTERYX at each site. Injection sites were classified as superficial (mucosa or submucosa), deep (muscularis propria or submucosa contiguous to muscularis), or subadventitial (between muscle and adventitia). Results: There was one area of transmural injection with ENTERYX adherent to the outside esophageal wall. There was no extravasated ENTERYX found in the mediastinum, or adherent to contiguous organs. In three areas the injection could not be assessed due to absence of material; these areas were counted as inadequate injections. Areas where there was both superficial and deep material were counted as deep injections. 85% of the injections were deep. The results are shown in Table 2, below. The average time to place 12 injections was 24 minutes. Conclusions: Using the device of FIG. 1A without fluoroscopy, ENTERYX can be consistently deposited into the deep esophageal wall with a high degree of accuracy in a minimal amount of time.

Location of ENTERYX Injections (%)

TABLE 2

| | Unable to | | Sub- | | |
|---|---|---|---|---|---|
| Total | Assess | Superficial | adventitial | Transmural | Deep |
| 60 | 3 (5%) | 1 (1.7%) | 4 (6.7%) | 1 (1.7%) | 51 (85%) |

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. An apparatus comprising:
an endoscope;
a flexible catheter adapted to be inserted via the endoscope to deliver a substance to a tissue from an internal aspect;
a penetrating member at a distal end of the catheter for penetrating the tissue, the penetrating member having a lumen in communication with the catheter, the lumen having an opening at a surface of the penetrating member to deliver the substance through the lumen; and
means for determining a location of the opening relative to the tissue.

2. The apparatus of claim 1, wherein the means for determining the location includes a mechanism to monitor a flow rate of a fluid through the catheter.

3. The apparatus of claim 2, wherein the mechanism to monitor the flow rate includes a flow-meter.

4. The apparatus of claim 2, wherein the fluid includes CO₂, air, or helium.

5. The apparatus of claim 2, wherein the catheter includes a first lumen for delivering the substance and a second lumen for conducting the fluid.

6. The apparatus of claim 2, further including a pump to pump the fluid through the catheter.

7. The apparatus of claim 1, wherein the means for determining the location includes a member to monitor a pressure proximate the opening.

8. The apparatus of claim 7, wherein the catheter includes at least two lumens, a first lumen used to monitor the pressure and a second lumen to deliver the substance.

9. The apparatus of claim 7, wherein the means to monitor the pressure includes a pressure sensor located proximate the opening of the penetrating member.

10. The apparatus of claim 1, wherein the means for determining the location includes a pressure gauge located at a proximal end of the catheter and the pressure gauge indicates a pressure of a fluid within the catheter.

11. The apparatus of claim 10, wherein the pressure is conducted through the fluid in the lumens of the catheter and penetrating member.

12. The apparatus of claim 10, wherein the fluid includes dimethyl sulfoxide.

13. The apparatus of claim 1, further comprising an injecting mechanism located at a proximal end of the catheter to deliver the substance through the penetrating member.

14. The apparatus of claim 13, wherein the injecting mechanism includes a syringe containing ethylene vinyl alcohol co-polymer.

15. The apparatus of claim 1, wherein the penetrating member and catheter are advanceable through the endoscope.

16. The apparatus of claim 1, wherein the penetrating member and catheter are advanceable alongside the endoscope.

17. The apparatus of claim 1, wherein the substance includes a therapeutic material.

18. The apparatus of claim 1, wherein the substance includes a suture.

19. The apparatus of claim 1, wherein the substance includes a clip, plicator, anchor, or plug.

20. An apparatus comprising:
a catheter having a first lumen for delivering a substance through the first lumen and having a second lumen for passing a fluid through the catheter; and a member to monitor a flow-rate of the fluid through the second lumen, wherein the flow-rate of the fluid through the second lumen is used to determine a location of a distal opening of the first lumen relative to the tissue.

21. The apparatus of claim 20, further including a penetrating member at a distal end of the catheter, the penetrating member having a first lumen in communication with the first lumen of the catheter.

22. The apparatus of claim 21, wherein the first lumen of the penetrating member is also in communication with the second lumen of the catheter.

23. The apparatus of claim 21, wherein the penetrating member includes a second lumen in communication with the second lumen of the catheter.

24. The apparatus of claim 23, wherein the first lumen of the penetrating member and the second lumen of the penetrating member each have an opening and the openings co-terminate proximate each other.

25. The apparatus of claim 23, wherein first lumen of the penetrating member and the second lumen of the penetrating member each have an opening and the openings terminate at different distances along the length of the penetrating member.

26. The apparatus of claim 23, wherein the penetrating member includes two separate penetrating members attached to the distal end of the catheter.

27. The apparatus of claim 23, wherein the penetrating member includes a single penetrating member having at least two separate lumens.

28. The apparatus of claim 20, wherein the first and second lumens are symmetrical in size.

29. The apparatus of claim 20, wherein the first and second lumens are asymmetrical in size.

30. The apparatus of claim 20, wherein the first and second lumens are coaxial.

31. The apparatus of claim 20, wherein the member to monitor the flow-rate includes a flow-meter.

32. The apparatus of claim 20, wherein when the member indicates that the flow-rate of the fluid through the second lumen is reduced, the distal opening is located within the tissue.

33. The apparatus of claim 20, The apparatus of claim 1, wherein the catheter is advanceable through an endoscope.

34. The apparatus of claim 20, wherein the penetrating member is advanceable alongside an endoscope.

35. The apparatus of claim 20, wherein the fluid can include CO₂, air, or helium.

36. A method comprising:
inserting a catheter into a body;
delivering a fluid through the catheter and monitoring a flow rate of the fluid out of an opening of the catheter;
inserting the catheter into a tissue; and
injecting a therapeutic material into the tissue through the catheter when it is determined that the flow rate is reduced, indicating that the opening of the catheter is located within the tissue.

37. The method of claim 36, further including determining by direct vision whether the therapeutic material is being injected too superficially.

38. The method of claim 36, wherein the fluid is delivered through a first lumen of the catheter and the therapeutic material is injected through a second lumen of the catheter.

39. The method of claim 36, wherein the therapeutic material includes ethylene vinyl alcohol co-polymer.

40. The method of claim 36, further including a penetrating member at a distal end of the catheter and communicating with one or more lumens of the catheter.

41. A method comprising:
inserting a catheter into a body;
delivering a fluid through a first lumen of the catheter;
monitoring a flow rate of the fluid or a pressure of the fluid within the first lumen;
inserting the catheter into a tissue; and
delivering a substance through the catheter when it is determined, by monitoring the flow rate or the pressure, that the catheter is properly located.

42. The method of claim 41, wherein the substance is delivered to the tissue.

43. The method of claim 41, wherein the substance is delivered to a location through the tissue.

44. The method of claim 41, wherein the substance is delivered through a second lumen of the catheter.

45. The method of claim 41, wherein the substance is delivered through the first lumen.

46. The method of claim 41, further including determining by direct vision whether the substance is in an appropriate layer of the tissue.

47. The method of claim 41, wherein the substance includes an ethylene vinyl alcohol co-polymer.

48. The method of claim 41, wherein the substance is a therapeutic material.

49. The method of claim 41, wherein the substance is a suture, a clip, a plicator, an anchor, or a plug.

50. A method comprising:
inserting a catheter into a body, the catheter having a penetrating member on a distal end of the catheter;
inserting a tip of the penetrating member into a tissue;
monitoring a pressure proximate the tip; and
delivering a substance through the penetrating member when the pressure indicates that the tip is properly located for the substance.

51. The method of claim 50, wherein monitoring the pressure includes sensing the pressure of a fluid located within a lumen of the catheter.

52. The method of claim 50, wherein monitoring the pressure includes using a pressure sensor proximate the tip.

53. The method of claim 50, wherein monitoring the pressure includes extending a probe through a lumen of the catheter.

54. The method of claim 50, wherein delivering includes delivering a therapeutic substance into the tissue.

55. The method of claim 50, wherein delivering includes delivering a clip, suture, plicator, anchor, or plug through the tissue or on the outside of the tissue.

56. The method of claim 50, further including determining by direct vision whether the distal opening is located appropriately.
